(19) United States
(12) Patent Application Publication
(10) Pub. No.: US 2010/0125173 A1
(43) Pub. Date: May 20, 2010

(54) Systems and Methods for Determining a Probability of Pulmonary Embolism in an Individual

(75) Inventor: Matteo Bottai, Columbia, SC (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/273,030

(22) Filed: Nov. 18, 2008

(51) Int. Cl. A61B 5/00; G06Q 50/00

(52) U.S. Cl. 600/300; 705/3

(57) ABSTRACT

In one embodiment, a method for determining a probability of pulmonary embolism in an individual is provided. The method includes collecting data about the individual that relates to a plurality of predefined variables, wherein each of the predefined variables has a predefined regression coefficient. A risk of the individual having a pulmonary embolism is predicted by applying a regression model to the data, the regression model being calculated by utilizing the predefined regression coefficients of the predefined variables.

### BACKGROUND

Clinical probability assessment is considered to be an important step in the diagnosis of pulmonary embolism. When considered individually, symptoms, signs, or common laboratory tests can have limited diagnostic power. Jointly, however, they can provide a more accurate assessment of the clinical probability of pulmonary embolism.

Recently, structured methods for determining probability of pulmonary embolism have been developed with the purpose of improving and easing the diagnostic approach. However, existing clinical models rest heavily on the interpretation of chest radiographs, which can require substantial medical expertise.

A simpler method of determining probability of pulmonary embolism based on clinical symptoms, signs, and the interpretation of an electrocardiogram would be beneficial. To facilitate use of such methods in clinical settings, easy-to-use systems for the computation of the clinical probability of pulmonary embolism would be particularly beneficial.

### SUMMARY

The present disclosure is generally directed to systems and methods for determining a probability of pulmonary embolism in an individual.

In one embodiment, a method for determining a probability of pulmonary embolism in an individual is provided. The method includes collecting data about the individual that relates to a plurality of predefined variables, wherein each of the predefined variables has a predefined regression coefficient. A risk of the individual having a pulmonary embolism is predicted by applying a regression model to the data, the regression model being calculated by utilizing the predefined regression coefficients of the predefined variables.

In another embodiment of the present disclosure, a computer implemented method for determining a probability of pulmonary embolism in an individual is provided. The method includes inputting data about the individual into a computer, the data relating to a plurality of predefined variables, wherein each of the predefined variables has a predefined regression coefficient. A risk of the individual having a pulmonary embolism is predicted by utilizing the computer to apply a regression model to the data, the regression model being calculated by utilizing the predefined regression coefficients of the predefined variables.

In still another embodiment of the present disclosure, a system for determining a probability of pulmonary embolism in an individual is provided. The system includes a computer program configured to receive data about the individual. The data relates to a plurality of predefined variables, wherein each of the predefined variables has a predefined regression coefficient. The program is configured to predict a risk of the individual having a pulmonary embolism by applying a regression model to the data, the regression model being calculated by utilizing the predefined regression coefficients of the predefined variables.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figure in which:

FIG. 1 illustrates the relationship between pretest (clinical) probability of pulmonary embolism and post test probability conditioned by the results of quantitative ELISA D-dimer test (A) and multidetector computed tomographic angiography (B); and

FIG. 2 illustrates the extent of scintigraphically detectable pulmonary vascular obstruction as a function of clinical probability in 440 patients with pulmonary embolism (line in box: 50^{th} percentile; limits of box: 25^{th} and 75^{th} percentile; whiskers: 10^{th} and 90^{th} percentile; parentheses, number of patients in each clinical probability category; P value <0.0001 by Kruskal-Wallis nonparametric test).

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The systems and methods discussed herein can be implemented using servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. The various computer systems that can be utilized with the present disclosure are not limited to any particular hardware architecture or configuration.

Embodiments of the methods and systems set forth herein can be implemented by one or more general-purpose or customized computing devices adapted in any suitable manner to provide desired functionality. The device(s) can be adapted to provide additional functionality complementary or unrelated to the present subject matter, as well. For instance, one or more computing devices can be adapted to provide desired functionality by accessing software instructions rendered in a computer-readable form.

When software is used, any suitable programming, scripting, or other type of language or combinations of languages can be used to implement the teachings contained herein. However, software need not be used exclusively, or at all. For example, some embodiments of the methods and systems set forth herein can also be implemented by hard-wired logic or other circuitry, including, but not limited to application-specific circuits. Of course, combinations of computer-executed software and hard-wired logic or other circuitry can be suitable, as well.

Embodiments of the methods disclosed herein can be executed by one or more suitable computing devices. Such system(s) can comprise one or more computing devices adapted to perform one or more embodiments of the methods disclosed herein. As noted above, such devices can access one or more computer-readable media that embody computer-readable instructions which, when executed by at least one computer, cause the at least one computer to implement one or more embodiments of the methods of the present subject matter. Additionally or alternatively, the computing device(s) can comprise circuitry that renders the device(s) operative to implement one or more of the methods of the present subject matter.

Any suitable computer-readable medium or media can be used to implement or practice the presently-disclosed subject matter, including, but not limited to, diskettes, drives, and other magnetic-based storage media, optical storage media, including disks (including CD-ROMS, DVD-ROMS, and variants thereof), flash, RAM, ROM, and other memory devices, and the like.

The present disclosure also can also utilize a relay of communicated data over one or more communications networks. It should be appreciated that network communications can comprise sending and/or receiving information over one or more networks of various forms. For example, a network can comprise a dial-in network, a local area network (LAN), wide area network (WAN), public switched telephone network (PSTN), the Internet, intranet or other type(s) of networks. A network can comprise any number and/or combination of hard-wired, wireless, or other communication links.

The present disclosure is generally directed to systems and methods for determining a probability of pulmonary embolism in an individual. A risk of pulmonary embolism for an individual is predicted by correlating data from the individual with a regression model. In some embodiments, the regression model is a multivariate logistic regression model. However, any suitable regression model as would be known in the art can be utilized. The model is calculated by utilizing predefined regression coefficients of predefined variables. The systems and methods described herein are based on the evaluation of relevant clinical symptoms and signs, which can include the interpretation of an electrocardiogram. Therefore, the systems and methods are applicable in any clinical context.

In one embodiment of the present disclosure, a multivariate logistic regression model was developed from a database of 1,100 patients with suspected pulmonary embolism, of whom 440 had the disease confirmed by angiography or autopsy findings. The model was validated in an independent sample of 400 patients with suspected pulmonary embolism.

The model utilized in the systems and methods described herein can include one or more of 16 variables of which 10 (older age, male sex, prolonged immobilization, history of deep vein thrombosis, sudden-onset dyspnea, chest pain, syncope, hemoptysis, unilateral leg swelling, electrocardiographic signs of acute cor pulmonale) are positively associated, and 6 (prior cardiovascular or pulmonary disease, orthopnea, high fever, wheezes, or crackles on chest auscultation) are negatively associated with pulmonary embolism.

Among various symptoms, sudden-onset dyspnea can be a strong predictor of pulmonary embolism. In terms of predictive accuracy, the systems and methods of the present disclosure outperform conventional methods for predicting pulmonary embolism. In addition, the systems and methods described herein perform equally well in inpatients and outpatients. Among patients with pulmonary embolism, there was a strong relation between the clinical probability predicted and the severity of embolism on lung scintigraphy—the higher the probability, the greater the extent of vascular obstruction.

Traditional methods of predicting pulmonary embolism require substantial medical expertise. The present disclosure includes variables that are negatively associated with pulmonary embolism, which gives the models greater flexibility and can explain why the systems and methods perform equally well in detecting and in ruling out pulmonary embolism. Also, instead of using a point-scale score proportional to the regression coefficients, the systems and methods of the present disclosure estimate the probability of pulmonary embolism directly from the sum of the regression coefficients. This allows prediction of the clinical probability as a continuous function and precise estimation of odds ratios.

In certain embodiments of the present disclosure, the relative complexity of the calculations described herein can be overcome by using software. For instance, in certain embodiments, dedicated software that permits online computation of clinical probability can be utilized.

In certain embodiments, the software can be very minimal in size, such as less than about 20 kilobytes in size, so as to be capable of use on various devices. For example, the systems and methods of the present disclosure can be implemented on desktop computers, laptop computers, personal digital assistants, mobile phones, and the like, such as other hand held devices, as would be known and understood in the art. In certain embodiments, software in accordance with the systems and methods of the present disclosure can be downloaded from the Internet to any suitable device.

The high positive and negative predicted values of the models described herein, irrespective of the prevalence of pulmonary embolism, suggests that the proposed models can be applied in evaluating patients from different populations with varying prevalence of the disease. The clinical probability predicted by the models described herein can be used by physicians as the pretest probability in calculating the post test probability of pulmonary embolism after appropriate objective testing.

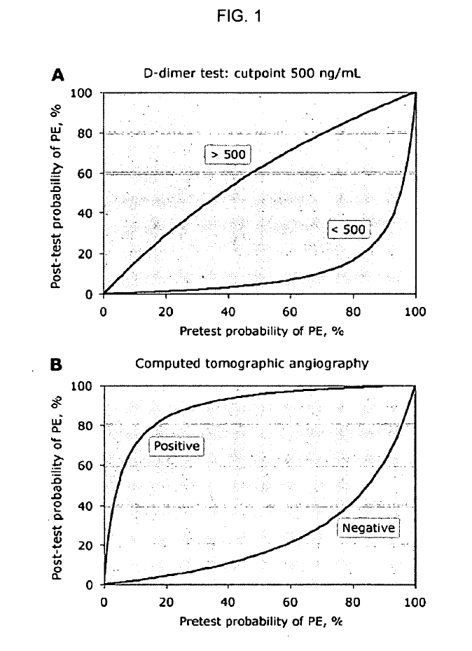

An example is given in FIG. 1, which shows the relationship between pretest and post test probability of pulmonary embolism conditioned by the results of quantitative D-dimer test or computed tomographic angiography (CTA). D-dimer tests or CTA can be utilized when there is a suspicion of pulmonary embolism. For the D-dimer test, a weighted sensitivity of about 98% and a weighted specificity of about 40% were utilized. For CTA, a weighted sensitivity of about 83% and a weighted specificity of about 96% were utilized.

This formal analysis indicates the following: (1) a D-dimer concentration of about 500 ng/ml or less with a pretest probability less than about 50% makes a diagnosis of pulmonary embolism very unlikely; (2) a D-dimer concentration greater than about 500 ng/ml does not modify the pretest probability and is, therefore, clinically irrelevant; (3) a negative CTA with a pretest probability of about 10% or less rules out clinically significant pulmonary embolism; (4) a positive CTA with a pretest probability of about 50% or greater makes a diagnosis of pulmonary embolism very likely; (5) when pretest probability and CTA results are discordant, the post test probability is neither sufficiently high nor sufficiently low to permit therapeutic decisions; under these circumstances further diagnostic testing is mandatory. The model of the present disclosure can provide physicians with a diagnostic edge when evaluating patients for suspected pulmonary embolism.

The present disclosure can be better understood with reference to the following examples.

### EXAMPLES

### Example 1

### Methods

### Sample of Patients

The sample comprised 1,100 patients who were referred to the Institute of Clinical Physiology (Pisa, Italy) for suspected pulmonary embolism between Nov. 1, 1991, and Dec. 31, 1999. Approximately 70% of patients were referred from the medical or surgical departments, and from the emergency ward of the city hospital; about 30% came from four peripheral hospitals in northwestern Tuscany. The suspicion of pulmonary embolism had been raised on the basis of the following: presence of symptoms such as unexplained dyspnea, chest pain, fainting, or hemoptysis; electrocardiographic or echocardiographic signs of acute right ventricular overload; arterial hypoxemia with respiratory alkalosis. None of the patients had undergone any objective testing for pulmonary embolism before entering the study. Presence or absence of pulmonary embolism was firmly established in all patients. Patients' baseline characteristics are reported in Example 2. All the patients were examined uniformly according to a standardized protocol that included clinical evaluation, perfusion lung scanning, and pulmonary angiography. The interpretation of lung scans and angiograms was done as would be understood by one of ordinary skill in the art. All the procedures, including pulmonary angiography, were performed in a dedicated diagnostic unit. The protocol was approved by the local ethics committee. Before angiography, an informed, written consent was obtained.

### Clinical Evaluation

Upon study entry, patients were examined by 1 of 12 chest physicians who served as on-call physicians 1 day a week. When interviewing the patients, care was taken to identify risk factors for pulmonary embolism and preexisting diseases that may mimic the clinical presentation of pulmonary embolism. In evaluating dyspnea, attention was paid to establish whether it was sudden or gradual in onset, or whether it was associated with orthopnea. Unilateral leg swelling with or without tenderness and redness of the skin was regarded as a sign suggestive of deep vein thrombosis. Electrocardiograms obtained within 24 hours before study entry were considered for evaluation by the physicians. Acute cor pulmonale was deemed present if one or more of the following abnormalities were identified: S wave in lead I and Q wave in lead III, each of an amplitude greater than 1.5 mm; with T-wave inversion in lead III (S₁Q₃T₃), S waves in lead I, II, and III, each of an amplitude greater than 1.5 mm (S₁S₂S₃); T-wave inversion in right precordial leads, transient right bundle branch block, and pseudoinfarction. If any of the above abnormalities were present in electrocardiograms taken before the onset of symptoms, they were disregarded. All clinical and laboratory data were recorded by the physicians on a standard form before any further objective testing. The data were stored in a computer for further analysis.

### Diagnostic Criteria for Pulmonary Embolism

The diagnosis of pulmonary embolism was based on angiography or autopsy documentation of pulmonary emboli. Criteria for excluding pulmonary embolism were a normal pulmonary angiogram, absence of pulmonary emboli at autopsy, or a normal perfusion scan. Pulmonary angiograms were not obtained in patients with normal scans because available data indicated that such a scintigraphic pattern alone makes a diagnosis of pulmonary embolism very unlikely. A 6-month clinical follow-up was pursued in patients with normal scans at inclusion. In patients with confirmed pulmonary embolism, a scintigraphic follow-up was obtained at 1 week, 1 month, and 1 year of diagnosis to assess the restoration of pulmonary perfusion.

### Pulmonary Angiography

Pulmonary cineangiograms were obtained according to standardized procedures within 24 hours of study entry. Initial filming was in the anteroposterior view, after having advanced the catheter into the main pulmonary artery of the lung that showed the greatest perfusion abnormalities on lung scanning. If there was doubt about the presence of filling defects, the appropriate vessel was selectively entered with a balloon-tipped catheter and angiograms were repeated by manual injection of contrast material. Pulmonary angiograms were examined by experienced physicians who were blinded to clinical information. Angiographic criteria for diagnosing pulmonary embolism included the identification of an embolus obstructing a vessel or the outline of an embolus within a vessel. In patients who died before angiography, the diagnosis was established at autopsy.

### Severity of Pulmonary Embolism

The extent of scintigraphically detectable pulmonary vascular obstruction was estimated at baseline as an index of disease severity. This analysis was performed by a nuclear medicine specialist, who was blinded to clinical information, according to a method originally validated against pulmonary angiography. Further details are provided in Example 2.

### Derivation of the Predictive Model

The sample prevalence for all the variables collected in the 1,100 patients was provided separately for patients with and those without pulmonary embolism. Age was the only continuous variable, and it was grouped into four classes based on the quartiles of its observed distribution (15-56, 57-67, 68-74, and 75-94 yr). The univariate relationship between patients' baseline characteristics and the diagnosis of pulmonary embolism was assessed by Fisher's exact test or by Mann-Whitney nonparametric test. Two-tailed P values less than 0.05 were considered statistically significant throughout. A logistic regression model was developed for the probability of having pulmonary embolism. Initially, all the baseline variables were included in the model. Then, they were removed one by one, if not statistically significant. If the removal caused large changes (0.10%) in the coefficients of any of the remaining variables, the removed variable was reintroduced into the model. In the model-building process, age and sex were considered known relevant predictors and were kept in the model regardless of their statistical significance. In the final model, however, all the variables included were statistically significant (Table 1). The area under the receiver operating characteristic (ROC) curve of the final model was reported, together with its 95% confidence interval (CI). All the analysis was performed on Stata (STATA 10; StataCorp, College Station, Tex.) and R software (http://www.r-project.org).

### Internal Validation of the Model

To estimate the predictive accuracy of the models of the present disclosure, when applied to a new set of patients, bootstrap resampling techniques were used. The area under the ROC curve was estimated from 1,000 bootstrap samples of size 1,100 that were randomly selected with replacement from the original 1,100-patient sample.

### External Validation of the Model

The predictive model, derived from the original 1,100-patient sample, was validated in an independent sample of 454 patients with suspected pulmonary embolism who were evaluated between Jan. 1, 2003, and Dec. 31, 2005. Fifty-four (12%) of them were excluded because of inability to obtain an informed consent (n=28), or documented contraindications to pulmonary angiography (n=26). The remaining 400 patients were managed according to the diagnostic protocol described above. The clinical probability of pulmonary embolism was estimated at bedside by one of seven residents in respiratory medicine by using the proposed software on palm computers. Clinical probability was assessed before any further objective testing. The management of the 54 patients who were excluded from the validation sample is described in the online supplement.

### Results

### Derivation Sample

The 1,100 patients in the derivation sample had a median age of 68 years (interquartile range [IQR], 57-75 yr); 45% of them were male, and 81% were hospitalized at the time of study entry. On the basis of angiography and autopsy findings, the prevalence of pulmonary embolism was 40%. The median extent of pulmonary vascular obstruction at baseline was 42% (IQR, 30-57%). Most of the patients with pulmonary embolism showed a nearly complete restoration of pulmonary perfusion, with 90% of them having a residual vascular obstruction of less than 15% at 1 year of diagnosis. Among the patients without pulmonary embolism, 242 had normal perfusion scans. None of these patients had symptomatic episodes of venous thromboembolism during a 6-month period of follow-up.

### Predictive Model

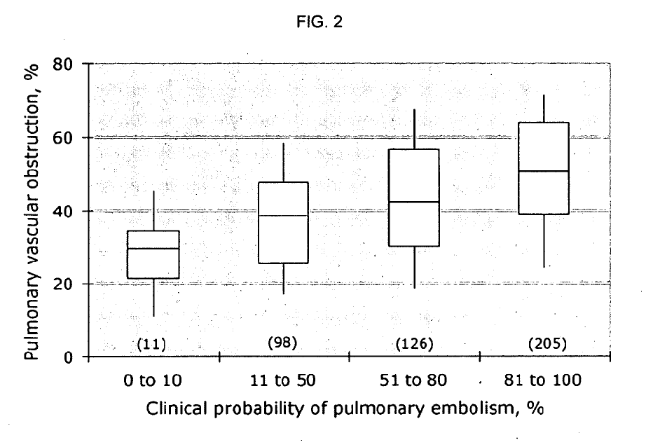

Sixteen variables were incorporated into a multivariate logistic regression model, of which 10 were positively and 6 were negatively associated with pulmonary embolism (Table 1). Variables associated with an increased likelihood of pulmonary embolism were as follows: older age, male sex, prolonged immobilization, history of deep vein thrombosis, sudden-onset dyspnea, chest pain, fainting (or syncope), hemoptysis, and electrocardiographic signs of acute cor pulmonale. Variables associated with a decreased likelihood of pulmonary embolism included prior cardiovascular or pulmonary disease, orthopnea, high fever, wheezes, or crackles on chest auscultation. The area under the ROC curve was 0.90 (95% CI, 0.88-0.91). The probability of pulmonary embolism can be calculated after adding all the applicable regression coefficients to the constant, as shown in the footnote of Table 1. For practical purposes, the probability of pulmonary embolism was grouped into four categories: slight (0 to about 10%), moderate (about 11 to about 50%), substantial (about 51 to about 80%), and high (about 81 to about 100%). The proportion of patients in each of the four probability categories and the relative prevalence of pulmonary embolism are reported in Table 2. Among the 440 patients with pulmonary embolism, there was a highly significant, positive relation between the clinical probability predicted by the model and the extent of scintigraphically detectable pulmonary vascular obstruction (FIG. 2).

### Internal Validation

The predictive model derived from the original 1,100-patient sample appeared to be accurate and parsimonious. The overall accuracy of the model, as measured by the ROC area, was validated based on 1,000 bootstrap samples. The area under the ROC curve on a sample of new independent patients was estimated to be 0.88.

### External Validation

The 400 patients in the validation sample had a median age of 70 years (IQR, 59-76 yr); 42% were male, and 71% were inpatients at the time of study entry. Further characteristics are given in Example 2. Pulmonary embolism was diagnosed by angiography in 165 (41%) of 400 patients. The median extent of pulmonary vascular obstruction at baseline was 40% (IQR, 27-56%). Of the 235 patients without pulmonary embolism, 83 had normal perfusion scans. None of them presented with symptomatic episodes of venous thromboembolism over a 6-month follow-up. The proportion of patients in each of the four probability categories and the relative prevalence of pulmonary embolism are reported in Table 3 for the whole sample, and separately for inpatients and outpatients. In the validation sample, the area under the ROC curve was 0.88 (95% CI, 0.84-0.91), which was consistent with the estimate from the internal validation. The prevalence of pulmonary embolism in outpatients (46%) was slightly but not significantly higher than in inpatients (39%, P value 5 0.18 by Fisher's test). There was no significant difference between inpatients and outpatients regarding the prevalence of pulmonary embolism in each of the four probability categories (Table 3). In the 165 patients with pulmonary embolism at inclusion, there was a highly significant, positive relation between the clinical probability predicted by the model and the severity of pulmonary embolism on the lung scan (P value, 0.001 by Kruskal-Wallis nonparametric test).

### Example 2

### Methods

### Clinical Evaluation

Upon study entry, patients were examined by one of twelve chest physicians who served as on-call physicians one day a week. When interviewing the patients, care was taken to identify risk factors for pulmonary embolism and pre-existing diseases which may mimic the clinical presentation of pulmonary embolism. Immobilization for longer than 3 consecutive days, any major surgical procedure, or any radiologically confirmed bone fracture of the lower extremities was considered risk factors if they occurred within 4 weeks prior to study entry. History of lower limb deep vein thrombosis, or any prior episode of pulmonary embolism were recorded if the patient had, at any time, documented episodes of deep vein thrombosis or pulmonary embolism which required anticoagulant therapy. Estrogen use was defined as use of estrogen-containing drugs within the past 3 months. Post-partum period was defined as the presence of pregnancy within the past 3 months.

Cardiovascular (coronary artery disease, arterial hypertension, heart failure, left heart valvular disease, cerebrovascular disease), pulmonary (chronic obstructive pulmonary disease, asthma, interstitial lung disease), endocrine (diabetes mellitus, thyroid dysfunction of any cause), or any other kind of nonmalignant diseases were recorded if documented any time prior to study entry. Neoplastic disease was recorded if there was evidence of clinically active malignancy with pathologic diagnosis within the past 3 months. Patients' baseline characteristics are reported in Table 4.

TABLE 4
Baseline characteristics of derivation sample

| | PE | | No PE | | |
|---|---|---|---|---|---|
| | (n = 440) | | (n = 660) | | |
| Characteristic | Number (%) or Median (IQR) | | | | P-value |
| Inpatients | 354 | (80) | 537 | (81) | 0.75 |
| Age (years) | 68 | (57-75) | 67 | (57-74) | 0.25 |
| Male sex | 205 | (47) | 290 | (44) | 0.39 |
| Risk factors | | | | | |
| Immobilization (>3 days) | 242 | (55) | 290 | (44) | 0.0004 |
| Deep vein thrombosis (ever) | 153 | (35) | 109 | (17) | <0.0001 |
| Recent surgery | 160 | (36) | 224 | (34) | 0.44 |
| Recent trauma | 78 | (18) | 86 | (13) | 0.04 |
| Post-partum | 2 | (0.5) | 10 | (1.5) | 0.14 |
| Estrogen use | 4 | (1) | 6 | (0.1) | 1.00 |
| Pre-existing diseases | | | | | |
| Cardiovascular | 128 | (29) | 273 | (41) | <0.0001 |
| Pulmonary | 37 | (8) | 132 | (20) | <0.0001 |
| Endocrine | 35 | (8) | 82 | (12) | 0.02 |
| Neoplastic | 70 | (16) | 92 | (14) | 0.39 |
| Symptoms | | | | | |
| Dyspnea (sudden onset) | 358 | (81) | 210 | (32) | <0.0001 |
| Dyspnea (gradual onset) | 14 | (3) | 143 | (22) | <0.0001 |
| Orthopnea | 3 | (0.7) | 62 | (9) | <0.0001 |
| Chest pain | 248 | (56) | 232 | (35) | <0.0001 |
| Fainting or syncope | 114 | (26) | 83 | (13) | <0.0001 |
| Hemoptysis | 31 | (7) | 25 | (4) | 0.02 |
| Cough | 48 | (11) | 112 | (17) | 0.005 |
| Palpitations | 100 | (23) | 117 | (18) | 0.04 |
| Signs | | | | | |
| Tachycardia (>100 beats/min) | 124 | (28) | 180 | (27) | 0.78 |
| Cyanosis | 44 | (10) | 57 | (9) | 0.46 |
| Hypotension (<90 mmHg) | 12 | (3) | 8 | (1) | 0.10 |
| Neck vein distention | 64 | (15) | 57 | (9) | 0.003 |
| Leg swelling (unilateral) | 101 | (23) | 63 | (10) | <0.0001 |
| Fever (>38° C.) | 28 | (6) | 180 | (27) | <0.0001 |
| Crackles | 72 | (16) | 181 | (27) | <0.0001 |
| Wheezes | 11 | (3) | 75 | (11) | <0.0001 |
| Pleural friction rub | 15 | (3) | 22 | (3) | 1.00 |
| Electrocardiogram | | | | | |
| Acute cor pulmonale | 200 | (45) | 54 | (8) | <0.0001 |

PE: pulmonary embolism;
IQR: interquartile range.

### Perfusion Lung Scanning

Perfusion lung scans were obtained after intravenous injection of human serum albumin microspheres labeled with 99 m Technetium (150 MBq), and were acquired by means of a large field gamma camera equipped with a high resolution, parallel-hole collimator, using a 20% symmetric window set over the 140 KeV photopeak. Images consisted of anterior, posterior, both lateral, and both posterior oblique views, with 500,000 counts per image. Lung scans were attributed to one of four predetermined categories: normal (no perfusion defects); near-normal (impressions caused by enlarged heart, hila, or mediastinum are seen on an otherwise normal scan); abnormal, suggestive of pulmonary embolism (single or multiple wedge-shaped perfusion defects); abnormal, not suggestive of pulmonary embolism (single or multiple perfusion defects other than wedge-shaped). Lung scans were read by experienced physicians who were blinded to clinical information.

### Pulmonary Angiography

Pulmonary cineangiograms were obtained according to standardized procedures within 24 hours of study entry. Initial filming was in the anteroposterior view, after having advanced the catheter into the main pulmonary artery of the lung which showed the greatest perfusion abnormalities on lung scanning. If there was a doubt about the presence of filling defects, the appropriate vessel was selectively entered with a balloon-tipped catheter and angiograms were repeated by manual injection of contrast material. Pulmonary angiograms were examined by experienced physicians who were blinded to clinical information. Angiographic criteria for diagnosing pulmonary embolism included the identification of an embolus obstructing a vessel or the outline of an embolus within a vessel. In patients who died before angiography, the diagnosis was established at autopsy.

### Severity of Pulmonary Embolism on Lung Scintigraphy

The extent of scintigraphically detectable pulmonary vascular obstruction at inclusion was measured as an index of the severity of pulmonary embolism. The cumulative distribution of the vascular obstruction index is reported in Table 5 along with the cumulative normal distribution. The difference between the observed distribution and the normal distribution was assessed by Kolmogorov test. Data refer to 440 patients with angiography or autopsy confirmed pulmonary embolism comprised in the derivation sample.

TABLE 5
Cumulative distribution of vascular obstruction index

| | |
|---|---|
| Obstruction index (%) | Cumulative distribution |

| From (≧) | To (<) | Observed | Normal | D |
|---|---|---|---|---|
| 4.5 | 12.2 | 0.03182 | 0.04133 | 0.00951 |
| 12.2 | 19.9 | 0.10455 | 0.09655 | 0.008 |
| 19.9 | 27.7 | 0.20682 | 0.19285 | 0.01397 |
| 27.7 | 35.4 | 0.35682 | 0.33234 | 0.02448 |
| 35.4 | 43.1 | 0.50909 | 0.50022 | 0.00887 |
| 43.1 | 50.8 | 0.64545 | 0.66805 | 0.0226 |
| 50.8 | 58.6 | 0.78636 | 0.80745 | 0.02109 |
| 58.6 | 66.3 | 0.87955 | 0.90363 | 0.02408 |
| 66.3 | 74.0 | 0.96364 | 0.95887 | 0.00477 |
| 74.0 | 81.7 | 1.00000 | 0.98502 | 0.01498 |

D = absolute difference between the observed cumulative distribution and the cumulative normal distribution. Kolmogorov test calculates lambda = Dmax * √n where n = number of observations.
For Dmax = 0.02448, lambda = 0.5135 and P(lambda) = 0.964

Based on the above, the conclusion was made that the sample of patients with pulmonary embolism is representative of the whole spectrum of severity of the disease, from minor to massive.

### Results

### Clinical Probability Assessment Among Different Raters

In the validation sample of 400 patients, the clinical probability of pulmonary embolism was rated by one of seven residents in respiratory medicine using the prediction model described in the text.

A logit-binomial regression model for grouped data was applied to test for differences in the predictive accuracy of the model among the seven raters. Results are reported in Table 6 and 7.

TABLE 6
Comparison between clinical probability and actual
prevalence of pulmonary embolism for seven different raters

Clinical probability %

| | | | |
|---|---|---|---|
| 0 to 10 | 11 to 50 | 51 to 80 | 81 to 100 |

| | | |
|---|---|---|
| Rater | PE/No. of patients | Total |

| | | | | | |
|---|---|---|---|---|---|
| 1 | 2/14 | 7/22 | 3/7 | 17/18 | 29/61 |
| 2 | 0/16 | 4/12 | 7/14 | 9/11 | 20/53 |
| 3 | 1/13 | 5/20 | 9/10 | 15/15 | 30/58 |
| 4 | 0/24 | 1/9 | 8/11 | 14/15 | 23/59 |
| 5 | 0/26 | 2/10 | 8/10 | 9/9 | 19/55 |
| 6 | 0/18 | 8/16 | 4/7 | 11/13 | 23/54 |
| 7 | 0/25 | 2/15 | 4/5 | 15/15 | 21/60 |
| Total | 3/136 | 29/104 | 43/64 | 90/96 | 165/400 |

PE: pulmonary embolism

TABLE 7
Odds ratios and 95% confidence interval (CI) of the clinical probability
of pulmonary embolism among seven different raters

| Rater | Odds ratio | 95% CI | P-value |
|---|---|---|---|
| 2 | 0.49 | 0.16-1.47 | 0.204 |
| 3 | 1.37 | 0.49-3.83 | 0.548 |
| 4 | 0.64 | 0.21-1.98 | 0.439 |
| 5 | 0.93 | 0.30-2.91 | 0.898 |
| 6 | 1.07 | 0.36-3.19 | 0.898 |
| 7 | 0.70 | 0.23-2.16 | 0.534 |

For odds ratios, rater 1 is the reference.
Test for “all the raters having the same probability”: P-value = 0.62

Based on the above, the conclusion was drawn that the predictive accuracy of the model does not differ among the seven raters.

Management of Patients Excluded from Validation Sample

The 54 patients who were excluded from the study were managed noninvasively by combining clinical probability with perfusion lung scan results. Pulmonary embolism was diagnosed when a clinical probability >50% was paired with a lung scan showing segmental perfusion defects. Pulmonary embolism was deemed absent on the basis of a normal scan or of a clinical probability ≦10% and a lung scan with perfusion defects other than segmental. Whenever required, lower limb ultrasonography was used to look for deep vein thrombosis.

Pulmonary embolism was diagnosed in 21 (39%) of these 54 patients. None of the patients in whom pulmonary embolism was deemed absent had symptomatic episodes of venous thromboembolism during follow-up.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure so further described in such appended claims.

TABLE 1
ESTIMATES FOR REGRESSION COEFFICIENTS, ODDS
RATIOS, AND 95% CONFIDENCE INTERVALS OF THE
PREDICTORS OF PULMONARY EMBOLISM

| Predictor | Coefficient | Odds Ratio | 95% CI |
|---|---|---|---|
| Age, yr | | | |
| 57-67 | 0.80 | 2.23 | 1.37-3.63 |
| 68-74 | 0.87 | 2.38 | 1.41-4.01 |
| ≧75 | 1.14 | 3.11 | 1.82-5.32 |
| Male sex | 0.60 | 1.82 | 1.27-2.61 |
| Risk factors | | | |
| Immobilization | 0.42 | 1.53 | 1.08-2.15 |
| Deep vein thrombosis (ever) | 0.64 | 1.90 | 1.23-2.95 |
| Preexisting diseases | | | |
| Cardiovascular | −0.51 | 0.60 | 0.41-0.88 |
| Pulmonary | −0.89 | 0.41 | 0.24-0.72 |
| Symptoms | | | |
| Dyspnea (sudden onset) | 2.00 | 7.38 | 5.18-10.51 |
| Orthopnea | −1.51 | 0.22 | 0.05-0.93 |
| Chest pain | 1.01 | 2.74 | 1.93-3.88 |
| Fainting or syncope | 0.66 | 1.93 | 1.25-2.98 |
| Hemoptysis | 0.93 | 2.52 | 1.19-5.35 |
| Signs | | | |
| Leg swelling (unilateral) | 0.80 | 2.23 | 1.35-3.70 |
| Fever > 38° C. | −1.47 | 0.23 | 0.13-0.40 |
| Wheezes | −1.20 | 0.30 | 0.14-0.66 |
| Crackles | −0.61 | 0.54 | 0.35-0.83 |
| Electrocardiogram | | | |
| Acute car pulmonale* | 1.96 | 7.11 | 4.66-10.87 |
| Constant | −3.43 | | |

Definition of abbreviation: CI = confidence interval.
*One or more of the following abnormalities: S₁Q₃T₃, S₁S₂S₃, negative T waves in right precordial leads, transient right bundle branch block, pseudoin-farction.
Calculation of the clinical probability of pulmonary embolism:
(1) Add all the coefficients that apply to a given patients and the constant (sum);
(2) the probability of pulmonary embolism equals [1 + exp(−sum)]⁻¹.

TABLE 2
COMPARISON BETWEEN CLINICAL PROBABILITY
AND ACTUAL PREVALENCE OF PULMONARY
EMBOLISM IN DERIVATION SAMPLE

| Clinical | No. of | | No. of | | 95% |
|---|---|---|---|---|---|
| Probability (%) | Patients | % | PEs | % | CI |
| 0-10 | 309 | 28 | 11 | 4 | 2-6 |
| 11-50 | 371 | 34 | 98 | 26 | 22-31 |
| 51-80 | 195 | 18 | 126 | 65 | 57-71 |
| 81-100 | 225 | 20 | 205 | 91 | 87-95 |

Definition of abbreviations:
CI = confidence interval;
PE = pulmonary embolism.

TABLE 3
COMPARISON BETWEEN CLINICAL PROBABILITY
AND ACTUAL PREVALENCE OF PULMONARY
EMBOLISM IN VALIDATION SAMPLE

| Clinical | All Patients | | | | Inpatients | | | | Outpatients | | | |
|---|---|---|---|---|---|---|---|---|---|---|---|---|
| Probability | (n = 400) | | | | (n = 284) | | | | (n = 116) | | | |
| (%) | Patients | % | PEs | % | Patients | % | PEs | % | Patients | % | PEs | % |
| 0-10 | 136 | 34 | 3 | 2 | 96 | 34 | 3 | 3 | 40 | 35 | 0 | 0* |
| 11-50 | 104 | 26 | 29 | 28 | 82 | 29 | 25 | 30 | 22 | 19 | 4 | 18^{†} |
| 51-80 | 64 | 16 | 43 | 67 | 43 | 15 | 26 | 60 | 21 | 18 | 17 | 81^{‡} |
| 81-100 | 96 | 24 | 90 | 94 | 63 | 22 | 57 | 90 | 33 | 28 | 33 | [symbol] |

Definition of abbreviation:
PE = pulmonary embolism.
*P = 0.555 compared with inpatients (Fisher's test).
^{†}P = 0.297 compared with inpatients (Fisher's test).
^{‡}P = 0.156 compared with inpatients (Fisher's test). [symbol]
= 0.091 compared with inpatients (Fisher's test).

What is claimed is:

1. A method for determining a probability of pulmonary embolism in an individual comprising:
collecting data about the individual that relates to a plurality of predefined variables, wherein each of the predefined variables has a predefined regression coefficient;
predicting a risk of the individual having a pulmonary embolism by applying a regression model to the data, the regression model being calculated by utilizing the predefined regression coefficients of the predefined variables.

2. The method of claim 1, wherein the plurality of predefined variables comprises at least one of the following: age, sex, prolonged immobilization, history of deep vein thrombosis, sudden-onset dyspnea, chest pain, syncope, hemoptysis, unilateral leg swelling, electrocardiographic signs of acute cor pulmanale, prior cardiovascular disease, prior pulmonary disease, orthopnea, high fever, wheezes, or crackles on chest auscultation.

3. The method of claim 1, wherein at least one of the predefined variables has a positive predefined regression coefficient indicating increased probability of pulmonary embolism.

4. The method of claim 1, wherein at least one of the predefined variables has a negative predefined regression coefficient indicating decreased probability of pulmonary embolism.

5. The method of claim 3, wherein at least one of the predefined variables having a positive predefined regression coefficient comprises age, sex, prolonged immobilization, history of deep vein thrombosis, sudden-onset dyspnea, chest pain, syncope, hemoptysis, unilateral leg swelling, or electrocardiographic signs of acute cor pulmanale.

6. The method of claim 3, wherein at least one of the predefined variables having a negative predefined regression coefficient comprises prior cardiovascular disease, prior pulmonary disease, orthopnea, high fever, wheezes, or crackles on chest auscultation.

7. The method of claim 1, wherein the plurality of predefined variables comprises age, sex, prolonged immobilization, history of deep vein thrombosis, sudden-onset dyspnea, chest pain, syncope, hemoptysis, unilateral leg swelling, electrocardiographic signs of acute cor pulmanale, prior cardiovascular disease, prior pulmonary disease, orthopnea, high fever, wheezes, or crackles on chest auscultation.

8. The method of claim 1, wherein the regression model comprises a multivariate logistic regression model.

9. The method of claim 1, wherein the plurality of predefined variables having predefined regression coefficients comprises sex and sudden-onset dyspnea, sudden-onset dyspnea having a higher predefined regression coefficient than sex.

10. The method of claim 1, wherein the plurality of predefined variables having predefined regression coefficients comprises sex and fever, fever having a lower predefined regression coefficient than sex.

11. A computer implemented method for determining a probability of pulmonary embolism in an individual comprising:
inputting data about the individual into a computer, the data relating to a plurality of predefined variables, wherein each of the predefined variables has a predefined regression coefficient;
predicting a risk of the individual having a pulmonary embolism by utilizing a computer to apply a regression model to the data, the regression model being calculated by utilizing the predefined regression coefficients of the predefined variables.

12. The method of claim 11, wherein the plurality of predefined variables comprises at least one of the following: age, sex, prolonged immobilization, history of deep vein thrombosis, sudden-onset dyspnea, chest pain, syncope, hemoptysis, unilateral leg swelling, electrocardiographic signs of acute cor pulmanale, prior cardiovascular disease, prior pulmonary disease, orthopnea, high fever, wheezes, or crackles on chest auscultation.

13. The method of claim 11, wherein at least one of the predefined variables has a positive predefined regression coefficient indicating increased probability of pulmonary embolism.

14. The method of claim 11, wherein at least one of the predefined variables has a negative predefined regression coefficient indicating decreased probability of pulmonary embolism.

15. The method of claim 13, wherein at least one of the predefined variables having a positive predefined regression coefficient comprises age, sex, prolonged immobilization, history of deep vein thrombosis, sudden-onset dyspnea, chest pain, syncope, hemoptysis, unilateral leg swelling, or electrocardiographic signs of acute cor pulmanale.

16. The method of claim 14, wherein at least one of the predefined variables having a negative predefined regression coefficient comprises prior cardiovascular disease, prior pulmonary disease, orthopnea, high fever, wheezes, or crackles on chest auscultation.

17. A system for determining a probability of pulmonary embolism in an individual comprising:
a computer program configured to receive data about the individual, the data relating to a plurality of predefined variables, wherein each of the predefined variables has a predefined regression coefficient, the program configured to predict a risk of the individual having a pulmonary embolism by applying a regression model to the data, the regression model being calculated by utilizing the predefined regression coefficients of the predefined variables.

18. The system of claim 17, wherein the plurality of predefined variables comprises at least one of the following: age, sex, prolonged immobilization, history of deep vein thrombosis, sudden-onset dyspnea, chest pain, syncope, hemoptysis, unilateral leg swelling, electrocardiographic signs of acute cor pulmanale, prior cardiovascular disease, prior pulmonary disease, orthopnea, high fever, wheezes, or crackles on chest auscultation.

19. The system of claim 17, wherein the computer program is designed to be operable on a handheld device.

20. The system of claim 17, wherein the handheld device comprises a mobile phone or a personal digital assistant.
